(19) United States
(12) Patent Application Publication
(10) Pub. No.: US 2009/0306067 A1
(43) Pub. Date: Dec. 10, 2009

(54) 2, 4-DIAMINOPYRIMIDIDE DERIVATES AND THEIR USE FOR THE TREATMENT OF CANCER

(76) Inventors: Harald Engelhardt, Ebreichsdorf (AT); Heinz Stadtmueller, Vienna (AT); Martin Steegmaier, Reutlingen (DE)

Correspondence Address: MICHAEL P. MORRIS;BOEHRINGER INGELHEIM USA CORPORATION, 900 RIDGEBURY RD, P. O. BOX 368, RIDGEFIELD, CT, 06877-0368, US

(21) Appl. No.: 12/296,829

(22) PCT Filed: Apr. 4, 2007
(86) PCT No.: PCT/EP2007/053327

§ 371 Date: Dec. 5, 2008

(30) Foreign Application Priority Data
Apr. 10, 2006 (EP) 06112414.5

(51) Int. Cl. A61K 31/538; C07D 487/04; A61K 31/4985; C07D 498/04; A61P 35/00

(52) U.S. Cl. 514/230.5; 544/295; 514/249; 544/105

(57) ABSTRACT

The present invention encompasses compounds of general formula (1) wherein Q and R¹ to R⁴ are defined as in claim 1, which are suitable for the treatment of diseases characterised by excessive or anomalous cell proliferation, and their use for preparing a pharmaceutical composition having the above-mentioned properties.

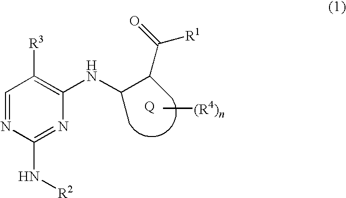

The present invention relates to new compounds of general formula (1)

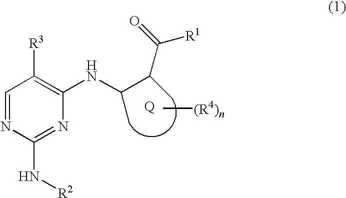

wherein the groups Q and R¹ to R⁴ have the meanings given in the claims and specification, the isomers thereof, processes for preparing these compounds and their use as medicaments.

### BACKGROUND TO THE INVENTION

Tumour cells wholly or partly elude regulation and control by the body and are characterised by uncontrolled growth. This is due on the one hand to the loss of control proteins such as for example RB, p16, p21 and p53 and also to the activation of so-called accelerators of the cell cycle, the cyclin-dependent kinases.

Studies in model organisms such as Schizosaccharomyces pombe, Drosophila melanogaster or Xenopus laevis as well as investigations in human cells have shown that the transition from the G2 phase to mitosis is regulated by the CDK1/cyclin B kinase (Nurse 1990, Nature 344: 503-508). This kinase, which is also known as “mitosis promoting factor” (MPF), phosphorylates and thereby regulates a plurality of proteins, such as e.g. nuclear lamina, kinesin-like motor proteins, condensins and Golgi Matrix Proteins, which play an important part in the breakdown of the nuclear coat, in centrosome separation, the structure of the mitotic spindle apparatus, chromosome condensation and breakdown of the Golgi apparatus (Nigg. E. 2001, Nat Rev Mol Cell Biol. 2(1):21-32). A murine cell line with a temperature-sensitive CDK-1 kinase mutant shows a rapid breakdown in CDK-1 kinase after temperature increase and a subsequent arrest in the G2/M phase (Th'ng et al., 1990, Cell. 63(2):313-24). The treatment of human tumour cells with inhibitors against CDK1/cyclin B, such as e.g. butyrolactone, leads to an arrest in the G2/M phase and subsequent apoptosis (Nishio, et al. 1996, Anticancer Res. 16 (6B):3387-95).

Moreover, the protein kinase Aurora B has also been described as having an essential function during entry into mitosis. Aurora B phosphorylates histone H3 on Ser10 and thereby initiates chromosome condensation (Hsu et al. 2000, Cell 102:279-91). A specific cell cycle arrest in the G2/M phase may, however, also be initiated e.g. by inhibition of specific phosphatases such as e.g. Cdc25C (Russell and Nurse 1986, Cell 45:145-53). Yeasts with a defective Cdc25 gene arrest in the G2 phase, whereas overexpression of Cdc25 leads to premature entry into the mitosis phase (Russell and Nurse, 1987, Cell 49:559-67). Moreover, an arrest in the G2/M phase may also be initiated by inhibition of specific motor proteins, the so-called kinesins such as for example Eg5 (Mayer et al., 1999, Science 286:971-4)), or by microtubuli stabilising or destabilising agents (e.g. colchicin, taxol, etoposide, vinblastine, vincristine) (Schiff and Horwitz 1980, Proc Natl Acad Sci USA 77:1561-5).

In addition to the cyclin-dependent and Aurora kinases the so-called polo-like kinases (PLK), a small family of serine/threonine kinases, also play an important role in the regulation of the eukaryotic cell cycle. Hitherto, the polo-like kinases PLK-1, PLK-2, PLK-3 and PLK-4 have been described in the literature. PLK-1 in particular has been found to play a central role in the regulation of the mitosis phase. PLK-1 is responsible for the maturation of the centrosomes, for the activation of phosphatase Cdc25C, as well as for the activation of the Anaphase Promoting Complex (Glover et al. 1998, Genes Dev. 12:3777-87; Qian et al. 2001, Mol Biol Cell. 12:1791-9). The injection of PLK-1 antibodies leads to a G2 arrest in untransformed cells, whereas tumour cells arrest during the mitosis phase (Lane and Nigg 1996, J. Cell Biol. 135:1701-13). Overexpression of PLK-1 has been demonstrated in various types of tumour, such as non-small-cell carcinoma of the lung, plate epithelial carcinoma, breast and colorectal carcinoma (Wolf et al. 1997, Oncogene 14:543-549; Knecht et al. 1999, Cancer Res. 59:2794-2797; Wolf et al. 2000, Pathol. Res. Pract. 196:753-759; Takahashi et al. 2003, Cancer Sci. 94:148-52). Therefore, this category of proteins also presents an interesting point of attack for therapeutic intervention in proliferative diseases (Liu and Erikson 2003, Proc Natl Acad Sci USA 100:5789-5794).

The resistance of many types of tumours requires the development of new drugs for combating tumours. The aim of the present invention is therefore to indicate new active substances which may be used for the prevention and/or treatment of diseases characterised by excessive or anomalous cell proliferation.

### DETAILED DESCRIPTION OF THE INVENTION

It has now been found that, surprisingly, compounds of general formula (1), wherein the groups Q, R¹ to R⁴ are defined as hereinafter, act as inhibitors of specific cell cycle kinases. Thus, the compounds according to the invention may be used for example for the treatment of diseases associated with the activity of specific cell cycle kinases and characterised by excessive or anomalous cell proliferation.

The present invention relates to compounds of general formula (1)

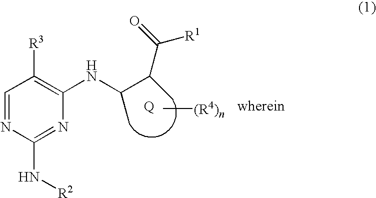

Q denotes 5-6 membered heteroaryl, and

R¹ denotes a group selected from among C₁₋₆alkyl, —NR^{c}R^{c} and —OR^{c}, or

R¹ together with a suitable R⁴ forms a 5-7 membered cycloaliphatic ring, which may optionally be substituted by one or more R⁵ and may optionally contain heteroatoms, selected from among N, O and S, and
R² denotes a group, optionally substituted by one or more R⁴, selected from among C₁₋₆alkyl, C₃₋₁₀cycloalkyl, 3-8 membered heterocycloalkyl, C₆₋₁₅aryl and 5-12 membered heteroaryl, and
R³ denotes a group selected from among hydrogen, halogen, —CN, —NO₂, C₁₋₄alkyl, C₁₋₄haloalkyl and —C(O)R^{c}, and
R⁴ and R⁵ each independently of one another denote a group selected from among R^{a}, R^{b} and R^{a} substituted by one or more identical or different R^{c} and/or R^{b}, and
n denotes 0, 1 or 2, and
each R^{a} is independently selected from among C₁₋₆alkyl, C₃₋₁₀cycloalkyl, C₄₋₁₆-cycloalkylalkyl, C₆₋₁₀aryl, C₇₋₁₆arylalkyl, 2-6 membered heteroalkyl, 3-8 membered heterocycloalkyl, 4-14 membered heterocycloalkylalkyl, 5-12 membered heteroaryl and 6-18 membered heteroarylalkyl, and
each R^{b} is a suitable group and each independently selected from among ═O, —OR^{c}, C₁₋₃-haloalkyloxy, —OCF₃, ═S, —SR^{c}, ═NR^{c}, ═NOR^{c}, —NR^{c}R^{c}, halogen, —CF₃, —CN, —NC, —OCN, —SCN, —NO₂, —S(O)R^{c}, —S(O)₂R^{c}, —S(O)₂OR^{c}, —S(O)NR^{c}R^{c}, —S(O)₂NR^{c}R^{c}, —OS(O)R^{c}, —OS(O)₂R^{c}, —OS(O)₂OR^{c}, —OS(O)₂NR^{c}R^{c}, —C(O)R^{c}, —C(O)OR^{c}, —C(O)NR^{c}R^{c}, —CN(R^{f})NR^{c}R^{c}, —CN(OH)R^{c}, —CN(OH)NR^{c}R^{c}, —OC(O)R^{c}, —OC(O)OR^{c}, —OC(O)NR^{c}R^{c}, —OCN(R^{f})NR^{c}R^{c}, —N(R^{f})C(O)R^{c}, —N(R^{f})C(S)R^{c}, —N(R^{f})S(O)₂R^{c}, —N(R^{f})C(O)OR^{c}, —N(R^{f})C(O)NR^{c}R^{c}, —[N(R^{f})C(O)]₂R^{c}, —N[C(O)]₂R^{c}, —N[C(O)]₂OR^{c}, —[N(R^{f})C(O)]₂OR^{c} and —N(R^{f})CN(R^{f})NR^{c}R^{c}, and
each R^{c} independently denotes hydrogen or a group optionally substituted by one or more identical or different R^{d} and/or R^{e} selected from among C₁₋₆alkyl, C₃₋₁₀cycloalkyl, C₄₋₁₁cycloalkylalkyl, C₆₋₁₀aryl, C₇₋₁₆arylalkyl, 2-6 membered heteroalkyl, 3-8 membered heterocycloalkyl, 4-14 membered heterocycloalkylalkyl, 5-12 membered heteroaryl and 6-18 membered heteroarylalkyl, and
each R^{d} independently denotes hydrogen or a group optionally substituted by one or more identical or different R^{e} and/or R^{f} selected from among C₁₋₆alkyl, C₃₋₈cycloalkyl, C₄₋₁₁cycloalkylalkyl, C₆₋₁₀aryl, C₇₋₁₆arylalkyl, 2-6 membered heteroalkyl, 3-8 membered heterocycloalkyl, 4-14 membered heterocycloalkylalkyl, 5-12 membered heteroaryl and 6-18 membered heteroarylalkyl, and
each R^{e} is a suitable group and each independently selected from among ═O, —OR^{f}, C₁₋₃₋haloalkyloxy, —OCF₃, ═S, —SR^{f}, ═NR^{f}, ═NOR^{f}, —NR^{f}R^{f}, halogen, —CF₃, —CN, —NC, —OCN, —SCN, —NO₂, —S(O)R^{f}, —S(O)₂R^{f}, —S(O)₂OR^{f}, —S(O)NR^{f}R^{f}, —S(O)₂NR^{f}R^{f}, —OS(O)R^{f}, —OS(O)₂R^{f}, —OS(O)₂OR^{f}, —OS(O)₂NR^{f}R^{f}, —C(O)R^{f}, —C(O)OR^{f}, —C(O)NR^{f}R^{f}, —CN(R^{g})NR^{f}R^{f}, —CN(OH)R^{f}, —C(NOH)NR^{f}R^{f}, —OC(O)R^{f}, —OC(O)OR^{f}, —OC(O)NR^{f}R^{f}, —OCN(R^{g})NR^{f}R^{f}, —N(R^{g})C(O)R^{f}, —N(R^{g})C(S)R^{f}, —N(R^{g})S(O)₂R^{f}, —N(R^{d})C(O)OR^{f}, —N(R^{g})C(O)NR^{f}R^{f}, and —N(R^{g})CN(R^{f})NR^{f}R^{f}, and
each R^{f} independently denotes hydrogen or a group optionally substituted by one or more identical or different R^{g} selected from among C₁₋₆alkyl, C₃₋₈cycloalkyl, C₄₋₁₁cycloalkylalkyl, C₆₋₁₀aryl, C₇₋₁₆arylalkyl, 2-6 membered heteroalkyl, 3-8 membered heterocycloalkyl, 4-14 membered heterocycloalkylalkyl, 5-12 membered heteroaryl and 6-18 membered heteroarylalkyl, and
each R^{g} independently denotes hydrogen, C₁₋₆alkyl, C₃₋₈cycloalkyl, C₄₋₁₁cycloalkylalkyl, C₆₋₁₀aryl, C₇₋₁₆arylalkyl, 2-6 membered heteroalkyl, 3-8 membered heterocycloalkyl, 4-14 membered heterocycloalkyl, 5-12 membered heteroaryl and 6-18 membered heteroarylalkyl,
optionally in the form of the tautomers, the racemates, the enantiomers, the diastereomers and the mixtures thereof, and optionally the pharmacologically acceptable salts thereof.

In one aspect the invention relates to compounds of general formula (1), wherein R³ denotes halogen or —CF₃.

In another aspect the invention relates to compounds of general formula (1), wherein R³ denotes —CF₃.

In another aspect the invention relates to compounds of general formula (1), wherein Q is selected from among thiophene, pyrrole, pyrazole and imidazole, optionally substituted by one or more R⁴.

In another aspect the invention relates to compounds of general formula (1), wherein Q is selected from among 1,2,3,4-tetrahydro-pyrrolo[1,2-a]pyrazine and 4,5,6,7-tetrahydro-pyrazolo[1,5-a]pyrazine, optionally substituted by one or more R⁵.

In another aspect the invention relates to compounds of general formula (1), wherein R² denotes a group, optionally substituted by one or more R⁴, selected from among C₆₋₁₅aryl and 5-12 membered heteroaryl.

In another aspect the invention relates to compounds of general formula (1) or the pharmaceutically effective salts thereof—for use as pharmaceutical compositions.

In another aspect the invention relates to compounds of general formula (1) —or the pharmaceutically effective salts thereof—for preparing a pharmaceutical composition with an antiproliferative activity.

In another aspect the invention relates to a pharmaceutical preparation, containing as active substance one or more compounds of general formula (1), or the pharmaceutically effective salts thereof, optionally in combination with conventional excipients and/or carriers.

In another aspect the invention relates to the use of compounds of general formula (1) for preparing a pharmaceutical composition for the treatment and/or prevention of cancer, infections, inflammations and autoimmune diseases.

In another aspect the invention relates to a pharmaceutical preparation comprising a compound of general formula (1), optionally in the form of the tautomers, the racemates, the enantiomers, the diastereomers and the mixtures thereof, and optionally the pharmacologically acceptable salts thereof and at least one other cytostatic or cytotoxic active substance different from formula (1).

### DEFINITIONS

As used herein the following definitions apply, unless stated otherwise.

By alkyl substituents are meant in each case saturated, unsaturated, straight-chain or branched aliphatic hydrocarbon groups (alkyl group) and this includes both saturated alkyl groups and unsaturated alkenyl and alkynyl groups. Alkenyl substituents are in each case straight-chain or branched, unsaturated alkyl groups, which have at least one double bond. By alkynyl substituents are meant in each case straight-chain or branched, unsaturated alkyl groups, which have at least one triple bond.

Heteroalkyl represents unbranched or branched aliphatic hydrocarbon chains which contain 1 to 3 heteroatoms, while each of the available carbon and heteroatoms in the heteroalkyl chain may optionally each be substituted independently and the heteroatoms independently of one another are selected from among O, N, P, PO, PO₂, S, SO and SO₂ (e.g. dimethylaminomethyl, dimethylaminoethyl, dimethylaminopropyl, diethylaminomethyl, diethylaminoethyl, diethylaminopropyl, 2-diisopropylaminoethyl, bis-2-methoxyethylamino, [2-(dimethylamino-ethyl)-ethyl-amino]-methyl, 3-[2-(dimethylamino-ethyl)-ethyl-amino]-propyl, hydroxymethyl, 2-hydroxyethyl, 3-hydroxypropyl, methoxy, ethoxy, propoxy, methoxymethyl, 2-methoxyethyl).

Haloalkyl refers to alkyl groups wherein one or more hydrogen atoms are replaced by halogen atoms. Haloalkyl includes both saturated alkyl groups and unsaturated alkenyl and alkynyl groups, such as for example —CF₃, —CHF₂, —CH₂F, —CF₂CF₃, —CHFCF₃, —CH₂CF₃, —CF₂CH₃, —CHFCH₃, —CF₂CF₂CF₃, —CF₂CH₂CH₃, —CF═CF₂, —CCl═CH₂, —CBr═CH₂, —CI═CH₂, —C≡C—CF₃, —CHFCH₂CH₃ and —CHFCH₂CF₃.

Halogen refers to fluorine, chlorine, bromine and/or iodine atoms.

By cycloalkyl is meant a mono- or polycyclic ring, wherein the ring system may be a saturated ring but also an unsaturated, non-aromatic ring or a spiro compound, which may optionally also contain double bonds, such as for example cyclopropyl, cyclopropenyl, cyclobutyl, cyclobutenyl, cyclopentyl, cyclopentenyl, cyclohexyl, cyclohexenyl, cycloheptanyl, cycloheptenyl, norbornyl, norbornenyl, indanyl, adamantyl, spiroheptanyl and spiro[4.2]heptanyl.

Cycloalkylalkyl includes a non-cyclic alkyl group wherein a hydrogen atom bound to a carbon atom is replaced by a cycloalkyl group.

Aryl relates to monocyclic or bicyclic rings with 6-12 carbon atoms such as for example phenyl and naphthyl.

Arylalkyl includes a non-cyclic alkyl group wherein a hydrogen atom bound to a carbon atom is replaced by an aryl group.

By heteroaryl are meant mono- or polycyclic rings having at least one aromatic ring which contain, instead of one or more carbon atoms, one or more heteroatoms, which may be identical or different, such as e.g. nitrogen, sulphur or oxygen atoms. Examples include furyl, furyl, thienyl, pyrrolyl, oxazolyl, thiazolyl, isoxazolyl, isothiazolyl, pyrazolyl, imidazolyl, triazolyl, tetrazolyl, oxadiazolyl, thiadiazolyl, pyridyl, pyrimidyl, pyridazinyl, pyrazinyl, triazinyl, indolyl, isoindolyl, benzofuranyl, benzothienyl, benzoxazolyl, benzothiazolyl, benzisoxazolyl, benzisothiazolyl, benzimidazolyl, indazolyl, isoquinolinyl, quinolinyl, quinoxalinyl, cinnolinyl, phthalazinyl, quinazolinyl and benzotriazinyl, indolizinyl, oxazolopyridinyl, imidazopyridinyl, naphthyridinyl, indolinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, phenoxazinyl, phenothiazinyl, pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, coumarinyl, isocoumarinyl, chromonyl, chromanonyl, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, tetrahydro-pyrrolopyrazine and tetrahydropyrazolopyrazine, pyrrolyl-N-oxide, pyridinyl-N-oxide, pyrimidinyl-N-oxide, pyridazinyl-N-oxide, pyrazinyl-N-oxide, quinolinyl-N-oxide, indolyl-N-oxide, indolinyl-N-oxide, isoquinolyl-N-oxide, quinazolinyl-N-oxide, quinoxalinyl-N-oxide, phthalazinyl-N-oxide, imidazolyl-N-oxide, isoxazolyl-N-oxide, oxazolyl-N-oxide, thiazolyl-N-oxide, indolizinyl-N-oxide, indazolyl-N-oxide, benzothiazolyl-N-oxide, benzimidazolyl-N-oxide, pyrrolyl-N-oxide, oxadiazolyl-N-oxide, thiadiazolyl-N-oxide, triazolyl-N-oxide, tetrazolyl-N-oxide, benzothiopyranyl-5-oxide and benzothiopyranyl-S,S-dioxide.

Heteroarylalkyl encompasses a non-cyclic alkyl group wherein a hydrogen atom bound to a carbon atom is replaced by a heteroaryl group.

Heterocycloalkyl relates to saturated or unsaturated, non-aromatic mono-, polycyclic or bridged polycyclic rings or spiro compounds comprising 3-12 carbon atoms, which carry heteroatoms, such as nitrogen, oxygen or sulphur, instead of one or more carbon atoms. Examples of such heterocyclyl groups are tetrahydrofuranyl, pyrrolidinyl, pyrrolinyl, imidazolidinyl, imidazolinyl, pyrazolidinyl, pyrazolinyl, piperidinyl, piperazinyl, indolinyl, isoindolinyl, morpholinyl, thiomorpholinyl, homomorpholinyl, homopiperidinyl, homopiperazinyl, homothiomorpholinyl, thiomorpholinyl-5-oxide, thiomorpholinyl-S,S-dioxide, tetrahydropyranyl, tetrahydrothienyl, homothiomorpholinyl-S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, tetrahydrothienyl-5-oxide, tetrahydrothienyl-S,S-dioxide, homothiomorpholinyl-5-oxide, 2-oxa-5-azabicyclo[2.2.1]heptane, 8-oxa-3-aza-bicyclo[3.2.1]octane, 3,8-diaza-bicyclo[3.2.1]octane, 2,5-diaza-bicyclo[2.2.1]heptane, 3,8-diaza-bicyclo[3.2.1]octane, 3,9-diaza-bicyclo[4.2.1]nonane and 2,6-diaza-bicyclo[3.2.2]nonane.

Heterocycloalkylalkyl relates to a non-cyclic alkyl group wherein a hydrogen atom bound to a carbon atom is replaced by a heterocycloalkyl group.

The following Examples illustrate the present invention without restricting its scope.

### Preparation of the Compounds According to the Invention

The compounds according to the invention may be prepared using the synthesis methods A to C described hereinafter. These methods are to be understood as being an illustration of the invention without restricting it to their content.

### Method A

### Step 1A

Intermediate compound III is prepared by substituting a leaving group LG, for example halogen, SCN, methoxy, preferably chlorine, in a heteroaromatic system I by a nucleophile II.

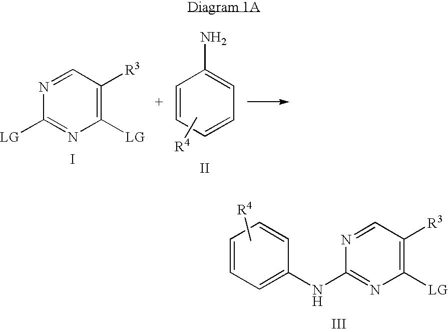

1 equivalent of compound I and 1 to 1.5 equivalents of compound II are stirred in a solvent, for example 1,4-dioxane, tetrahydrofuran, N,N-dimethylformamide or N,N-dimethylacetamide. At a temperature of 15 to 25° C., 2 to 2.5 equivalents of a base, for example potassium carbonate, sodium carbonate, caesium carbonate, N-ethyl-N,N-diisopropylamine or triethylamine are added. The reaction mixture is stirred for a further 12 to 72 h at a temperature of 15 to 25° C. Then the solvent is distilled off and the residue is mixed with water, which has been adjusted to a pH between 1-4 with an inorganic acid, for example hydrochloric acid or sulphuric acid. This mixture is extracted two to three times with an organic solvent, for example diethyl ether, ethyl acetate or dichloromethane. The combined organic extracts are dried and the solvent is distilled off. The residue is purified by chromatography.

### Step 2A

The preparation of the end compound V is carried out by substitution of a leaving group LG, for example halogen, SCN, methoxy, preferably chlorine, in a heteroaromatic system III by a nucleophile IV.

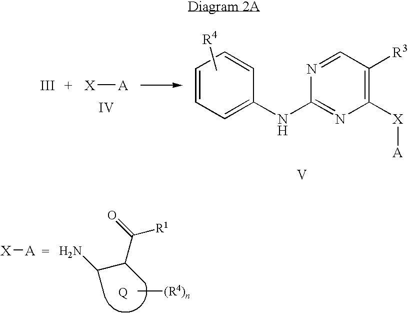

1 equivalent of compound III and 1 to 3 equivalents of compound IV are stirred in a solvent, for example 1,4-dioxane, N,N-dimethylformamide, N,N-dimethylacetamide or N-methyl-2-pyrrolidinone. At a temperature of 15 to 40° C., 1 to 2 equivalents of an inorganic acid, for example sulphuric acid or hydrochloric acid, are added. The reaction mixture is stirred for a further 12 to 72 h at a temperature of 20 to 100° C. Then the solvent is distilled off and the residue is purified by chromatography.

### Method B

### Step 1B

The intermediate compound VII is prepared by substitution of a leaving group LG, for example halogen, SCN, methoxy, preferably chlorine, in a heteroaromatic system I by a nucleophile VI.

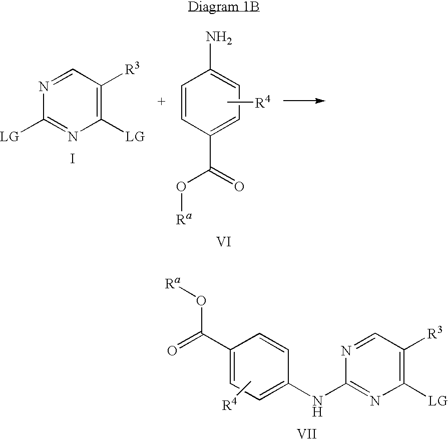

1 equivalent of compound I and 1 to 1.5 equivalents of the compound VI are stirred in a solvent, for example 1,4-dioxane, tetrahydrofuran, N,N-dimethylformamide or N,N-dimethylacetamide. At a temperature of 15 to 25° C., 2 to 2.5 equivalents of a base, for example potassium carbonate, sodium carbonate, caesium carbonate, potassium hydrogen phosphate, N-ethyl-N,N-diisopropylamine or triethylamine, are added. The reaction mixture is stirred for a further 2 to 8 h at a temperature of 50 to 120° C. The reaction mixture is mixed with water, which has been adjusted to a pH of 8 to 9 with an inorganic base, for example sodium hydrogen carbonate or potassium carbonate. This mixture is extracted two to three times with an organic solvent, for example diethyl ether or ethyl acetate. The combined organic extracts are dried and the solvent is distilled off. The residue is purified by chromatography or repeated crystallisation.

### Step 2B

The intermediate compound VIII is prepared by substitution of a leaving group LG, for example halogen, SCN, methoxy, preferably chlorine, in a heteroaromatic system VII by a nucleophile IV.

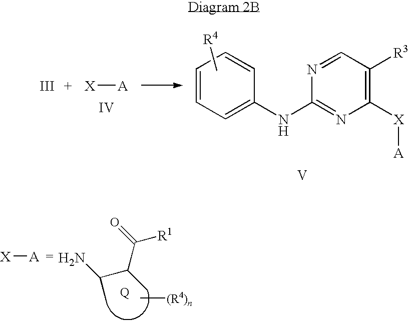

1 equivalent of the compound VII and 1 to 1.5 equivalents of the compound IV are stirred in a solvent, for example 1,4-dioxane, N,N-dimethylformamide, N,N-dimethylacetamide or N-methyl-2-pyrrolidinone. At a temperature of 15 to 40° C., 0.2 to 1 equivalent of an acid, for example sulphuric acid or hydrochloric acid, are added. The reaction mixture is stirred for a further 12 to 72 h at a temperature of 20 to 100° C. The reaction mixture is stirred into water and the precipitate formed is filtered off and dried. The precipitate may be purified by chromatography or crystallisation or used in the next step as the crude product.

### Step 3B

Compounds VIII wherein the group R denotes hydrogen may be used directly for preparing the end compounds X, by reacting a compound VIII with a compound IX. Compounds VIII having a group R which does not represent hydrogen are converted beforehand by hydrolysis or similar methods known to the skilled man into compounds wherein R═H,

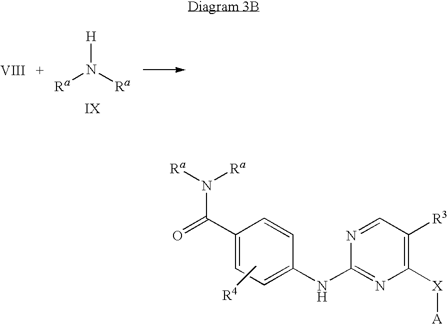

1 equivalent of the compound VIII, 1 to 1.5 equivalents of the compound IX and 1 to 3 equivalents of a base, for example triethylamine or ethyldiisopropylamine, are stirred in a solvent, for example 1,4-dioxane, N,N-dimethylformamide, N,N-dimethylacetamide or N-methyl-2-pyrrolidinone. At a temperature of 15 to 25° C., 1 to 1.5 equivalents of a coupling reagent, for example N,N-dicyclohexylcarbodiimide, N,N-diisopropylcarbodiimide, O-(benzotriazol-1-yl)-N,N,N′,N′-tetramethyluronium tetrafluoroborate or 1-(3-N,N-dimethylaminopropyl)-3-ethylcarbodiimide are added. The reaction mixture is stirred for a further 4 to 24 h at a temperature of 15 to 25° C. Then the solvent is distilled off and the residue is purified by chromatography.

### Method C

### Step 1C

The intermediate compound XI is prepared by substitution of a leaving group LG, for example halogen, SCN, methoxy, preferably chlorine, in a heteroaromatic system I by a nucleophile IV.

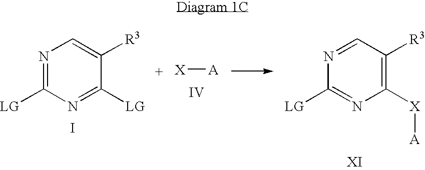

1 equivalent of the compound I and 1 to 3 equivalents of a base, for example triethylamine or ethyldiisopropylamine, are stirred in a solvent, for example 1,4-dioxane, tetrahydrofuran, N,N-dimethylformamide or N,N-dimethylacetamide. At a temperature of −60 to 0° C., 0.8 to 1.5 equivalents of a compound IV are added. The reaction mixture is stirred for a further 12 to 72 h at a temperature of 15 to 25° C. Then the solvent is distilled off and the residue is purified by chromatography.

### Step 2C

The end compound V is prepared by substitution of a leaving group LG, for example halogen, SCN, methoxy, preferably chlorine, in a heteroaromatic system XI by a nucleophile II.

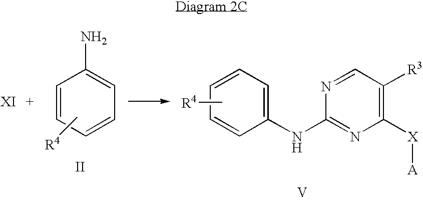

1 equivalent of the compound XI and 1 to 1.5 equivalents of the compound II are stirred in a solvent, for example 1,4-dioxane, N,N-dimethylformamide, N,N-dimethylacetamide or N-methyl-2-pyrrolidinone. At a temperature of 15 to 40° C. 1 to 2 equivalents of an acid, for example sulphuric acid or hydrochloric acid, are added. The reaction mixture is stirred for a further 12 to 72 h at a temperature of 20 to 100° C. Then the solvent is distilled off and the residue is purified by chromatography.

### Chromatography

For medium pressure chromatography (MPLC) silica gel made by Millipore (Granula Silica Si-60A 35-70 μm) or C-18 RP-silica gel made by Macherey Nagel (|Polygoprep 100-50 C18) is used.

For high pressure chromatography columns made by Waters (XTerra Prep. MS C18, 5 μM, 30*100 mm or Symmetry C18, 5 μm, 19*100) are used.

### Mass Spectroscopy/UV Spectrometer

These data are generated using an HPLC-MS apparatus (high performance liquid chromatography with mass detector) made by Agilent. The apparatus is designed so that a diode array detector (G1315B obtained from Agilent) and a mass detector (1100 LS-MSD SL; G1946D; Agilent) are connected in series after the chromatography (column: Zorbax SB-C8, 3.5 μm, 2.1*50, Agilent).

The apparatus is operated with a flow of 0.6 mL/min. For a separation process a gradient is run through within 3.5 min (gradient at the start: 95% water and 5% acetonitrile; gradient at the finish: 5% water and 95% acetonitrile; in each case 0.1% formic acid is added to each solvent).

### Method 1

### 2-(4-carboxy-2-methoxy-phenylamino)-4-chloro-5-trifluoromethyl-pyrimidine

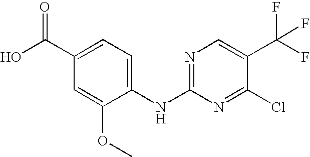

### a) 2-(4-benzyloxycarbonyl-2-methoxy-phenylamino)-4-chloro-5-trifluoromethyl-pyrimidine

2 g (9.22 mmol) 2,4-dichloro-5-trifluoromethylpyrimidine are dissolved in 4 mL dioxane and combined with 6 g (18.43 mmol) caesium carbonate and 2.16 g (7.36 mmol) benzyl 4-amino-3-methoxy-benzoate (WO9825901). This suspension is stirred for 30 h at 100° C. The suspension is combined with 50 mL each of dichloromethane and methanol and filtered to remove the insoluble matter. The solvent is eliminated in vacuo and the residue is purified by column chromatography. The carrier material used is silica gel and the eluant is a mixture consisting of 85% cyclohexane and 15% ethyl acetate.

Yield: 1.03 g

UV max: 320 nm

MS (ESI): 438/440 (M+H)⁺Cl distribution

436/438 (M−H)⁻Cl distribution

b) 2-(4-carboxy-2-methoxy-phenylamino)-4-chloro-5-trifluoromethyl-pyrimidine 1 g (2.28 mmol) 2-(4-benzyloxycarbonyl-2-methoxy-phenylamino)-4-chloro-5-trifluoromethyl-pyrimidine are dissolved in 50 mL THF and combined with 100 mg palladium hydroxide. The reaction mixture is stirred for 16 h at 20° C. and 4 bar hydrogen pressure. Then the catalyst is filtered off and the solvent is eliminated in vacuo.

Yield: 0.76 g

UV max: 288 nm

MS (ESI): 346/348 (M−H)⁻Cl distribution

Analogously to this process 2-[4-(4-benzyloxycarbonyl-piperazin-1-yl)-phenylamino]-4-chloro-5-trifluoromethyl-pyrimidine is prepared.

UV max: 298 nm

MS (ESI): 522/524 (M+H)⁺Cl distribution

### Method 2

### 8-amino-2-ethyl-3,4-dihydro-2H-pyrrolo[1,2-a]pyrazin-1-one

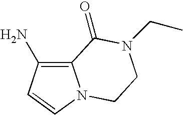

a) ethyl 3-tert-butoxycarbonylamino-1H-pyrrole-2-carboxylate 2.5 g (13.12 mmol) ethyl 3-amino-1H-pyrrole-2-carboxylate hydrochloride are dissolved in 4 mL dichloromethane and combined with 3.41 mL (19.67 mmol) N-ethyldiisopropylamine. 4.34 g (19.67 mmol) Boc-anhydride is dissolved in 7 mL dichloromethane and metered in over 8 h at ambient temperature using an injection pump. After 24 h the reaction mixture is diluted with dichloromethane and extracted with 10% potassium hydrogen sulphate solution. The organic phase is dried with magnesium sulphate and the solvent is eliminated in vacuo. The crude product is purified by column chromatography. The carrier material used is C18-RP-silica gel and a gradient is run through which consists of 80% water and 20% acetonitrile at the starting point and 30% water and 70% acetonitrile at the finishing point. 0.2% formic acid is added to each of the two eluants. The product fractions are combined and the solvent is eliminated using a freeze-drying apparatus.

Yield: 1.55 g

### b) ethyl 1-(2-bromo-ethyl)-3-tert-butoxycarbonylamino-1H-pyrrole-2-carboxylate

1 g (3.93 mmol) ethyl 3-tert-butoxycarbonylamino-1H-pyrrole-2-carboxylate are dissolved in 20 mL dimethylsulphoxide and combined with 1.1 g (20 mmol) potassium hydroxide. After this mixture has been stirred for 1 h at ambient temperature, 3.4 mL (39.33 mmol) 1,2-dibromoethane are added. After 16 h another 1.1 g (20 mmol) potassium hydroxide and 3.4 mL (39.33 mmol) 1,2-dibromoethane are added and the mixture is left for a further 24 h with stirring. The reaction mixture is combined with 300 mL water and extracted 3 times with 100 mL ethyl acetate. The organic phase is dried on magnesium sulphate and the solvent is eliminated in vacuo.

Yield: 1.35 g

### c) 8-amino-2-ethyl-3,4-dihydro-2H-pyrrolo[1,2-a]pyrazin-1-one

500 mg (1.38 mmol) ethyl 1-(2-bromo-ethyl)-3-tert-butoxycarbonylamino-1H-pyrrole-2-carboxylate is dissolved in 15 mL of a 70% aqueous ethylamine solution and the mixture is stirred for 48 h at 60° C. Then the reaction mixture is diluted with saturated sodium hydrogen carbonate solution and extracted with ethyl acetate. The organic phase is dried on magnesium sulphate and the solvent is eliminated in vacuo. This residue is combined with 5 mL of an isopropanolic hydrochloric acid solution (5 mol/L) and stirred for 12 h at ambient temperature. Then the solvent is eliminated in vacuo.

Yield: 209 mg

MS (ESI): 180 (M+H)⁺

The following compounds are prepared analogously:

| | MS (ESI) |
|---|---|
| | (M + H)⁺ |
| 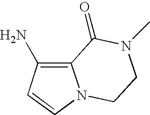 | 166 |
| 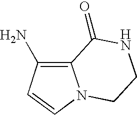 | 152 |
| 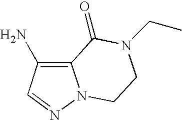 | 181 |

### Method 3

### 3-pyrrolidin-1-yl-cyclobutylamine

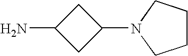

### a) tert-butyl (3-benzyloxy-cyclobutyl)-carbamate

9.28 g (45 mmol) 3-benzyloxy-cyclobutanecarboxylic acid (Organic Letters, 6(11), 1853-1856, 2004) are suspended in 80 mL dry tert-butanol and combined with 5.1 g (50 mmol) triethylamine and 13.8 g (50 mmol) phosphoric acid diphenylesterazide. The reaction mixture is stirred for 20 h under reflux conditions. The solvent is eliminated in vacuo and the residue is taken up in dichloromethane. The organic phase is washed 3 times with 2 N sodium hydroxide solution, dried on sodium sulphate and the dichloromethane is eliminated in vacuo. The crude product is recrystallised from acetonitrile (1 g crude product: 5 ml acetonitrile).

Yield: 5.98 g

MS (ESI): 178 (M+H-boc)⁺Boc cleaving in mass detector

### b) tert-butyl (3-hydroxy-cyclobutyl)-carbamate

2.77 g (10 mmol) tert-butyl (3-benzyloxy-cyclobutyl)-carbamate are suspended in 100 mL methanol and combined with 200 mg palladium hydroxide. The reaction mixture is stirred for 5 h at 45° C. and 45 bar hydrogen pressure. Then the catalyst is filtered off and the solvent is eliminated in vacuo. The residue is taken up in chloroform and washed 3 times with aqueous sodium hydrogen carbonate solution. The organic phase is dried on magnesium sulphate and the solvent is eliminated in vacuo.

Yield: 1.53 g

MS (ESI): 188 (M+H)⁺

### c) tert-butyl (3-tosyl-cyclobutyl)-carbamate

18.7 g (100 mmol) tert-butyl (3-hydroxy-cyclobutyl)-carbamate and 12.1 g (120 mmol) triethylamine are placed in 500 mL chloroform. 20.5 g (105 mmol) tosyl chloride, which is dissolved in 150 mL chloroform, is added dropwise to this solution at 0° C. with stirring. Then the reaction mixture is allowed to come up to 20° C. and stirred for a further 2 h. The organic phase is washed successively with water, with dilute hydrochloric acid, with sodium hydrogen carbonate solution and again with water. The organic phase is dried on magnesium sulphate and the solvent is eliminated in vacuo.

Yield: 28.3 g

MS (ESI): 342 (M+H)⁺

### d) tert-butyl (3-pyrrolidin-cyclobutyl)-carbamate

34.1 g (100 mmol) tert-butyl (3-tosyl-cyclobutyl)-carbamate are dissolved in 750 mL pyrrolidine, and a catalytic amount of DMAP is added. The reaction mixture is stirred for 20 h under reflux conditions. The pyrrolidine is eliminated in vacuo, the residue is taken up in 500 mL ethyl acetate and washed twice with saturated sodium hydrogen carbonate solution. The organic phase is dried on magnesium sulphate and the solvent is eliminated in vacuo. The crude product consists—as in all analogous reactions—of a mixture of 2 isomeric compounds which are separated by column chromatography. The stationary phase used is silica gel and the eluant is dichloromethane, to which 9% of a mixture of 90% methanol and 10% saturated aqueous ammonia solution have been added.

All the substances that elute first are designated as follows:

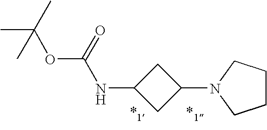

Yield product A: 1 g

R_{f} value (silica gel; dichloromethane:methanol:conc. aqueous ammonia=90:9:1)=0.62

All the substances that elute second are designated as follows:

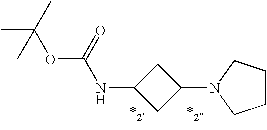

Yield product C: 2 g

R_{f} value (silica gel; dichloromethane:methanol:conc. aqueous ammonia=90:9:1)=0.53

### (*1′,*1″)-3-pyrrolidin-1-yl-cyclobutylamine

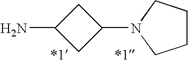

1 g (4.17 mmol) tert-butyl (3-pyrrolidin-cyclobutyl)-carbamate (product A from precursor) are stirred in 20 mL of a 2 N aqueous hydrochloric acid solution for 2 h at 40° C. Then the solvent is eliminated in vacuo and the residue is recrystallised from ethanol.

Yield: 0.43 g

MS (ESI): 141 (M+H)⁺

The following compound is prepared analogously to this process.

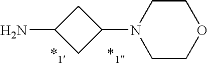

MS (ESI): 157 (M+H)⁺

### (*2′,*2″)-3-pyrrolidin-1-yl-cyclobutylamine

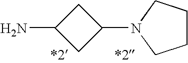

1 g (4.17 mmol) tert-butyl (3-pyrrolidin-cyclobutyl)-carbamate (product C from precursor) is stirred in 20 mL of a 2 N aqueous hydrochloric acid solution for 2 h at 40° C. Then the solvent is eliminated in vacuo and the residue is recrystallised from ethanol.

Yield: 0.43 g

MS (ESI): 141 (M+H)⁺

The following compound is prepared analogously to this process:

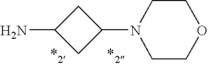

MS (ESI): 157 (M+H)⁺

### Method 4

### 4-(4-amino-cyclohexyl)-morpholine

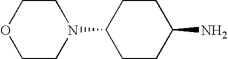

### a) dibenzyl-(4-morpholino-4-yl-cyclohexyl)-amine

3.9 g (30 mmol)) 4-dibenzylamino-cyclohexanone are dissolved in 100 mL dichloromethane and stirred with 3.9 g (45 mmol) morpholine and 9.5 g (45 mmol) sodiumtriacetoxy-borohydride for 12 h at ambient temperature. Then water and potassium carbonate are added, the organic phase is separated off, dried and the solvent is eliminated in vacuo. The crude product is purified by column chromatography. The carrier material used is silica gel and the eluant is ethyl acetate, to which 10% of a mixture of 90% methanol and 10% saturated aqueous ammonia solution have been added. The suitable fractions are evaporated down in vacuo.

Yield: 6.6 g cis-isomer

2g trans-isomer.

### b) trans-4-morpholin-4-yl-cyclohexylamine

7.2 g (16.4 mmol) trans-dibenzyl-4-morpholine-cyclohexylamine are dissolved in 100 mL MeOH and hydrogenated on 1.4 g Pd/C (10%) at 30-50° C. The solvent is eliminated in vacuo and the residue is crystallised from ethanol and concentrated HCl.

Yield: 3.9 g

Melting point: 312° C.

The following compound is prepared analogously to Method 4:

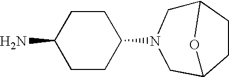

MS (ESI): 211 (M+H)⁺

### Method 5

### (R)-2-pyrrolidin-1-yl-propylamine

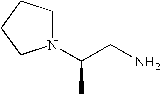

### a) (R)-2-pyrrolidin-1-yl-propionamide

2 g (16.06 mmol) R-alaninamine hydrochloride, 6.67 g (16.08 mmol) potassium carbonate and 8 mg (0.05 mmol) potassium iodide are suspended in 50 mL acetonitrile and then the mixture is combined with 1.92 mL (16.08 mmol) 1,4-dibromobutane. This reaction mixture is stirred for 14 h under reflux conditions. 100 mL of 1 N hydrochloric acid and 100 mL dichloromethane are added to the reaction mixture. The organic phase is separated off and discarded. The aqueous phase is made basic with sodium hydroxide solution and extracted 3 times with dichloromethane. The organic phases are combined, dried and freed from the solvent in vacuo.

Yield: 1.31 g

MS (ESI): 143 (M+H)⁺

### b) (R)-2-pyrrolidin-1-yl-propylamine

Under a nitrogen atmosphere 31.65 mL of 1 M lithium aluminium hydride solution (THF) are taken and combined at 0° C. with 1 g (7.03 mmol) (R)-2-pyrrolidin-1-yl-propionamide, which is dissolved in 2 mL THF. The reaction mixture is stirred for 48 h at 50° C., then combined with 100 mL methanol and then with the same amount of dichloromethane while cooling with ice. About 25 g silica gel are added to this mixture and the solvent is eliminated in vacuo. This silica gel is applied to a suction filter which has previously been loaded with about 75 g silica gel. The suction filter is washed batchwise with a total of 500 mL of a mixture of dichloromethane, methanol and aqueous concentrated ammonia (90:9:1). Most of the solvent is eliminated under a vacuum of 200 mbar and a sump temperature of approx. 50° C. The residue is purified by distillation.

Yield: 160 mg

MS (ESI): 129 (M+H)⁺

The following compound is prepared analogously to this process:

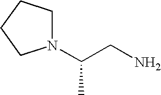

MS (ESI): 129 (M+H)⁺

### Example 1

### 2-[2-methoxy-4-(2-pyrrolidin-1-yl-ethylcarbamoyl)-phenylamino]-4-(2-ethyl-1-oxo-1,2,3,4-tetrahydro-pyrrolo[1,2-a]pyrazin-8-ylamino)-5-trifluoromethyl-pyrimidine

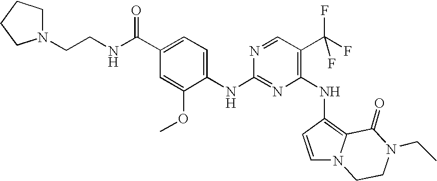

33 mg (0.09 mmol) of 2-(4-carboxyamino-2-methoxy-phenylamino)-4-chloro-5-trifluoromethyl-pyrimidine (Method 1) are dissolved in 100 μL N-methyl-2-pyrrolidinone and combined with 61 mg (0.14 mmol; content approx. 40%) 8-amino-2-ethyl-3,4-dihydro-2H-pyrrolo[1,2-a]pyrazin-1-one (Method 2). 15 μL of a 4 M solution of HCl (0.06 mmol) in 1,4-dioxane are metered into this reaction mixture. After 1 h at 100° C. the reaction mixture is stirred into 50 mL of an aqueous 1 N hydrochloric acid. The precipitate is filtered off and dried in vacuo. 34 mg (0.07 mmol) of this precipitate, 50 μL (0.31 mmol) N-ethyldiisopropylamine, 27 mg (0.08 mmol) O-(benzotriazol-1-yl)-N,N,N′,N′-tetramethyluronium-tetrafluoroborate and 13 μL (0.1 mmol) 1-(2-aminoethyl)-pyrrolidine are dissolved in 4 mL dichloromethane. After 1.5 h at ambient temperature the solvent is eliminated in vacuo. The crude product is purified by column chromatography. The carrier material used is C18-RP-silica gel and a gradient is run through which consists of 90% water and 10% acetonitrile at the starting point and of 40% water and 60% acetonitrile at the finishing point. 0.2% formic acid is added to each of the two eluants.

Yield: 23 mg

UV max: 322 nm

MS (ESI): 587 (M+H)⁺

### Examples 2-21

The following compounds are prepared by an analogous method to that described in Example 1.

The preparation of 2-(4-carboxyamino-phenylamino)-4-chloro-5-trifluoromethyl-pyrimidine is described in Method 1. The corresponding aniline is described in Method 2 or may be obtained commercially. The amine used to prepare the amide is commercially obtainable or is described in one of methods 3-5.

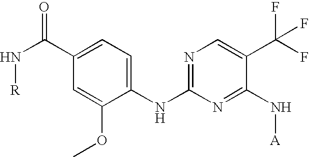

| | | | UV max | MS (ESI) |
|---|---|---|---|---|
| # | A | R | [nm] | (M + H)+ |
| 2 | 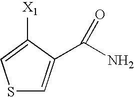 | 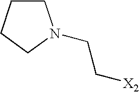 | 286, 315 | 550 |
| 3 | 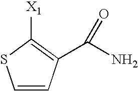 | 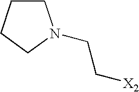 | 285, 325 | 550 |
| 4 | 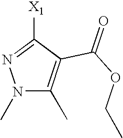 | 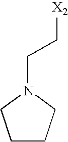 | 320, 286 | 591 |
| 5 | 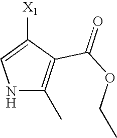 | 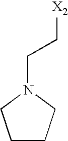 | 286, 314 | 576 |
| 6 | 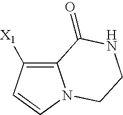 | 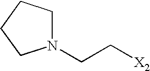 | 317 | 559 |
| 7 | 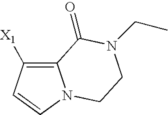 | 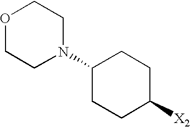 | 320 | 657 |
| 8 | 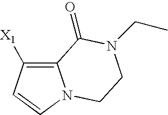 | 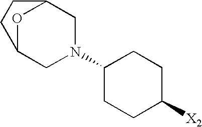 | 320 | 683 |
| 9 | 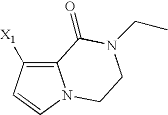 | 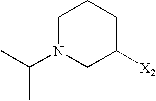 | 320 | 615 |
| 10 | 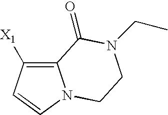 | 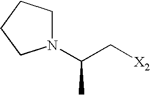 | 315 | 601 |
| 11 | 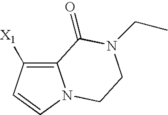 | 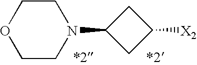 | 322 | 629 |
| 12 | 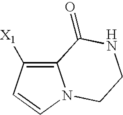 | 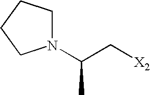 | 320 | 573 |
| 13 | 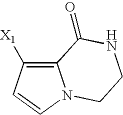 | 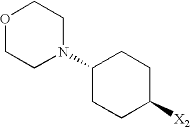 | 320 | 629 |
| 14 | 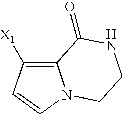 | 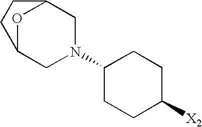 | 320 | 655 |
| 15 | 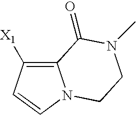 | 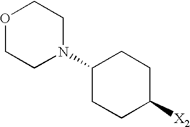 | 320 | 643 |
| 16 | 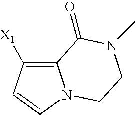 | 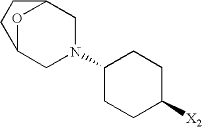 | 321 | 669 |
| 17 | 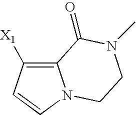 | 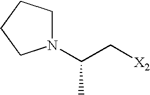 | 320 | 587 |
| 18 | 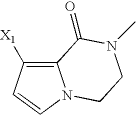 | 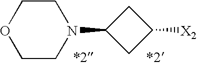 | 322 | 615 |
| 19 | 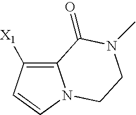 | 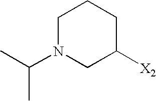 | 322 | 601 |
| 20 | 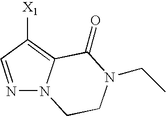 | 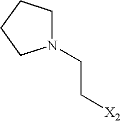 | | 588 |
| 21 | 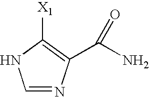 | 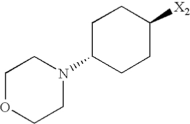 | | 604 |

### Example 22

### 2-(2-methoxy-4-piperazin-1-yl-phenylamino)-4-(2-ethyl-1-oxo-1,2,3,4-tetrahydro-pyrrolo 1,2-a]pyrazin-8-ylamino)-5-trifluoromethyl-pyrimidine

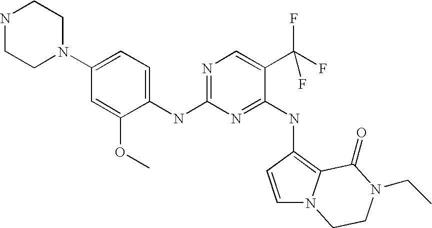

126 mg (0.21 mmol) 2-[4-(4-benzyloxycarbonyl-piperazin-1-yl)-phenylamino]-4-chloro-5-trifluoromethyl-pyrimidine are dissolved in 0.1 ml N-methyl-2-pyrrolidinone, then combined with 50 mg (0.21 mmol) 8-amino-2-ethyl-3,4-dihydro-2H-pyrrolo[1,2-a]pyrazin-1-one (Method 2) and with 25 μL (0.1 mmol) dioxanic hydrochloric acid. This reaction mixture is stirred for 1.5 h at 100° C. After 1 h at 100° C. the reaction mixture is stirred into 50 mL of an aqueous 1 N hydrochloric acid. The precipitate is filtered off and dried in vacuo. 113 mg (0.17 mmol) of the above-mentioned intermediate product are dissolved in 40 mL DMF and mixed with an amount of distilled water such that precipitation is only just avoided. 30 mg palladium on charcoal are added to this solution and it is hydrogenated at 7 bar hydrogen pressure and 20° C. for 3 h. The catalyst is filtered off and the solvent is eliminated in vacuo. The residue is purified by column chromatography. The carrier material used is C18-RP-silica gel and a gradient is run through which consists of 95% water and 5% acetonitrile at the starting point and 5% water and 95% acetonitrile at the finishing point. 0.2% formic acid is added to each of the eluants. The suitable fractions are freeze-dried. The residue is dissolved in acetonitrile and combined with 2 mL of a 1 M hydrochloric acid solution. Then the solvent is eliminated in vacuo. The substance is obtained as the dihydrochloride.

Yield: 56 mg

UV max: 258, 322 nm

MS (ESI): 531 (M+H)⁺

### Example 23

### 2-(2-methoxy-4-piperazin-1-yl-phenylamino)-4-(1-oxo-1,2,3,4-tetrahydro-pyrrolo[1,2-a]pyrazin-8-ylamino)-5-trifluoromethyl-pyrimidine

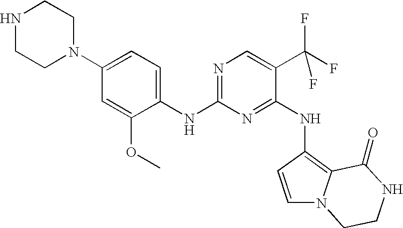

This compound is prepared analogously to Example 22.

UV max: 254, 322 nM

MS (ESI): 503 (M+H)⁺

The following Examples describe the biological activity of the compounds according to the invention without restricting the invention to these Examples.

The activity of the compounds according to the invention on various kinases, for example on serine-threonine kinase PLK-1, was determined by in vitro kinase assays with recombinantly produced protein. In this assay the compounds exhibit a good to very good effect on PLK1, i.e. for example an IC50 value of less than 1 μmol/L, usually less than 0.1 μmol/L.

### Example PLK-1 Kinaseassay

Recombinant human PLK1 enzyme linked to GST at its N-terminal end is isolated from insect cells infected with Baculovirus (Sf21). Purification is carried out by affinity chromatography on glutathione sepharose columns.

4×10⁷ Sf21 cells (Spodoptera frugiperda) in 200 ml of Sf-900 II Serum free insect cell medium (Life Technologies) are seeded in a spinner flask. After 72 hours' incubation at 27° C. and 70 rpm, 1×10⁸ Sf21 cells are seeded in a total of 180 ml medium in a new spinner flask. After another 24 hours, 20 ml of recombinant Baculovirus stock suspension are added and the cells are cultivated for 72 hours at 27° C. at 70 rpm. 3 hours before harvesting, okadaic acid is added (Calbiochem, final concentration 0.1 μM) and the suspension is incubated further. The cell number is determined, the cells are removed by centrifuging (5 minutes, 4° C., 800 rpm) and washed 1× with PBS (8 g NaCl/l, 0.2 g KCl/l, 1.44 g Na₂HPO₄/l, 0.24 g KH₂PO4/l). After centrifuging again the pellet is flash-frozen in liquid nitrogen. Then the pellet is quickly thawed and resuspended in ice-cold lysis buffer (50 mM HEPES pH 7.5, 10 mM MgCl₂, 1 mM DTT, 5 μg/ml leupeptin, 5 μg/ml aprotinin, 100 μM NaF, 100 μM PMSF, 10 mM β-glycerolphosphate, 0.1 mM Na₃VO₄, 30 mM 4-nitrophenylphosphate) to give 1×10⁸ cells/17.5 ml. The cells are lysed for 30 minutes on ice. After removal of the cell debris by centrifugation (4000 rpm, 5 minutes) the clear supernatant is combined with glutathione sepharose beads (1 ml resuspended and washed beads per 50 ml of supernatant) and the mixture is incubated for 30 minutes at 4° C. on a rotating board. Then the beads are washed with lysis buffer and the recombinant protein is eluted from the beads with 1 ml eluting buffer/ml resuspended beads (eluting buffer: 100 mM Tris/HCl pH=8.0, 120 mM NaCl, 20 mM reduced glutathione (Sigma G-4251), 10 mM MgCl₂, 1 mM DTT). The protein concentration is determined by Bradford Assay.

### Assay

The following components are combined in a well of a 96-well round-bottomed dish (Greiner bio-one, PS Microtitre plate No. 650101):
10 μl of the compound to be tested in variable concentrations (e.g. beginning at 300 μM, and dilution to 1:3) in 6% DMSO, 0.5 mg/ml casein (Sigma C-5890), 60 mM β-glycerophosphate, 25 mM MOPS pH=7.0, 5 mM EGTA, 15 mM MgCl₂, 1 mM DTT
20 μl substrate solution (25 mM MOPS pH=7.0, 15 mM MgCl₂, 1 mM DTT, 2.5 mM EGTA, 30 mM β-glycerophosphate, 0.25 mg/ml casein)
20 μl enzyme dilution (1:100 dilution of the enzyme stock in 25 mM MOPS pH=7.0, mM MgCl₂, 1 mM DTT)
10 μl ATP solution (45 μM ATP with 1.11×10⁶ Bq/ml gamma-P33-ATP).

The reaction is started by adding the ATP solution and continued for 45 minutes at 30° C. with gentle shaking (650 rpm on an IKA Schüttler MTS2). The reaction is stopped by the addition of 125 μl of ice-cold 5% TCA per well and incubated on ice for at least 30 minutes. The precipitate is transferred by harvesting onto filter plates (96-well microtitre filter plate: UniFilter-96, GF/B; Packard; No. 6005177), then washed four times with 1% TCA and dried at 60° C. After the addition of 35 μl scintillation solution (Ready-Safe; Beckmann) per well the plate is sealed shut with sealing tape and the amount of P33 precipitated is measured with the Wallac Betacounter. The measured data are evaluated using the standard Graphpad software (Levenburg-Marquard Algorhythmus).

The anti-proliferative activity of the compounds according to the invention is determined in the cytotoxicity test on cultivated human tumour cells and/or in a FACS analysis, for example on HeLa S3 cells. In both test methods the compounds exhibit good to very good activity, i.e. for example an EC50 value in the HeLa S3 cytotoxicity test of less than 5 μmol/L, generally less than 1 μmol/L.

### Measurement of Cytotoxicity on Cultivated Human Tumour Cells

To measure cytotoxicity on cultivated human tumour cells, cells of cervical carcinoma tumour cell line HeLa S3 (obtained from American Type Culture Collection (ATCC)) are cultivated in Ham's F12 Medium (Life Technologies) and 10% foetal calf serum (Life Technologies) and harvested in the log growth phase. Then the HeLa S3 cells are placed in 96-well plates (Costar) at a density of 1000 cells per well and incubated overnight in an incubator (at 37° C. and 5% CO2), while on each plate 6 wells are filled with medium alone (3 wells as the medium control, 3 wells for incubation with reduced AlamarBlue reagent). The active substances are added to the cells in various concentrations (dissolved in DMSO; DMSO final concentration: 0.1%) (in each case as a triple measurement). After 72 hours incubation 20 μl AlamarBlue reagent (AccuMed International) are added to each well, and the cells are incubated for a further 5-7 hours. As a control, 20 μl reduced AlamarBlue reagent is added to each of 3 wells (AlamarBlue reagent, which is autoclaved for 30 min). After incubation the colour change of the AlamarBlue reagent in the individual wells is determined in a Perkin Elmer fluorescence spectrophotometer (excitation 530 nm, emission 590 nm, slits 15, integrate time 0.1). The amount of AlamarBlue reagent reacted represents the metabolic activity of the cells. The relative cell activity is calculated as a percentage of the control (HeLa S3 cells without inhibitor) and the active substance concentration which inhibits the cell activity by 50% (IC50) is derived. The values are calculated from the average of three individual measurements—with correction of the dummy value (medium control).

### FACS Analysis

Propidium iodide (PI) binds stoichiometrically to double-stranded DNA, and is thus suitable for determining the proportion of cells in the G1, S, and G2/M phase of the cell cycle on the basis of the cellular DNA content. Cells in the G0 and G1 phase have a diploid DNA content (2N), whereas cells in the G2 or mitosis phase have a 4N DNA content.

For PI staining, for example, 1×10⁶ HeLa S3 cells are seeded onto a 75 cm2 cell culture flask, and after 24 h either 0.1% DMSO is added as control or the substance is added in various concentrations (in 0.1% DMSO). The cells are incubated for 24 h with the substance or with DMSO before the cells are washed 2× with PBS and then detached with trypsin/EDTA. The cells are centrifuged (1000 rpm, 5 min, 4° C.), and the cell pellet is washed 2× with PBS before the cells are resuspended in 0.1 ml PBS. Then the cells are fixed with 80% ethanol for 16 hours at 4° C. or alternatively for 2 hours at −20° C. The fixed cells are centrifuged (1000 rpm, 5 min, 4° C.), washed with PBS and then centrifuged again. The cell pellet is resuspended in 2 ml 0.25% Triton X-100 in PBS, and incubated on ice for 5 min before 5 ml PBS are added and the mixture is centrifuged again. The cell pellet is resuspended in 350 μl PI staining solution (0.1 mg/ml RNase A (Sigma, No. R-4875), 10 μg/ml prodium iodide (Sigma, No. P-4864) in 1×PBS). The cells are incubated for 20 min in the dark with the staining buffer before being transferred into sample measuring containers for the FACS scan. The DNA measurement is carried out in a Becton Dickinson FACS Analyzer, with an argon laser (500 mW, emission 488 nm), and the DNA Cell Quest Programme (BD). The logarithmic PI fluorescence is determined with a band-pass filter (BP 585/42). The cell populations in the individual cell cycle phases are quantified using the ModFit LT Programme made by Becton Dickinson.

The compounds according to the invention are also tested accordingly on other tumour cells. For example, these compounds are effective on carcinomas of all kinds of tissue (e.g. breast (MCF7); colon (HCT116), head and neck (FaDu), lung (NCI-H460), pancreas (BxPC-3), prostate (DU145)), sarcomas (e.g. SK-UT-1B), leukaemias and lymphomas (e.g. HL-60; Jurkat, THP-1) and other tumours (e.g. melanomas (BRO), gliomas (U-87MG)) and could be used for such indications. This is evidence of the broad applicability of the compounds according to the invention for the treatment of all kinds of tumour types.

On the basis of their biological properties the new compounds of general formula (1), their isomers and the physiologically acceptable salts thereof are suitable for treating diseases characterised by excessive or anomalous cell proliferation.

Such diseases include for example: viral infections (e.g. HIV and Kaposi's sarcoma); inflammatory and autoimmune diseases (e.g. colitis, arthritis, Alzheimer's disease, glomerulonephritis and wound healing); bacterial, fungal and/or parasitic infections; leukaemias, lymphomas and solid tumours (e.g. carcinomas and sarcomas), skin diseases (e.g. psoriasis); diseases based on hyperplasia which are characterised by an increase in the number of cells (e.g. fibroblasts, hepatocytes, bones and bone marrow cells, cartilage or smooth muscle cells or epithelial cells (e.g. endometrial hyperplasia)); bone diseases and cardiovascular diseases (e.g. restenosis and hypertrophy).

For example, the following cancers may be treated with compounds according to the invention, without being restricted thereto: brain tumours such as for example acoustic neurinoma, astrocytomas such as pilocytic astrocytomas, fibrillary astrocytoma, protoplasmic astrocytoma, gemistocytary astrocytoma, anaplastic astrocytoma and glioblastoma, brain lymphomas, brain metastases, hypophyseal tumour such as prolactinoma, HGH (human growth hormone) producing tumour and ACTH producing tumour (adrenocorticotropic hormone), craniopharyngiomas, medulloblastomas, meningeomas and oligodendrogliomas; nerve tumours (neoplasms) such as for example tumours of the vegetative nervous system such as neuroblastoma sympathicum, ganglioneuroma, paraganglioma (pheochromocytoma, chromaffinoma) and glomus-caroticum tumour, tumours on the peripheral nervous system such as amputation neuroma, neurofibroma, neurinoma (neurilemmoma, Schwannoma) and malignant Schwannoma, as well as tumours of the central nervous system such as brain and bone marrow tumours; intestinal cancer such as for example carcinoma of the rectum, colon, anus, small intestine and duodenum; eyelid tumours such as basalioma or basal cell carcinoma; pancreatic cancer or carcinoma of the pancreas; bladder cancer or carcinoma of the bladder; lung cancer (bronchial carcinoma) such as for example small-cell bronchial carcinomas (oat cell carcinomas) and non-small cell bronchial carcinomas such as plate epithelial carcinomas, adenocarcinomas and large-cell bronchial carcinomas; breast cancer such as for example mammary carcinoma such as infiltrating ductal carcinoma, colloid carcinoma, lobular invasive carcinoma, tubular carcinoma, adenocystic carcinoma and papillary carcinoma; non-Hodgkin's lymphomas (NHL) such as for example Burkitt's lymphoma, low-malignancy non-Hodgkin's lymphomas (NHL) and mucosis fungoides; uterine cancer or endometrial carcinoma or corpus carcinoma; CUP syndrome (Cancer of Unknown Primary); ovarian cancer or ovarian carcinoma such as mucinous, endometrial or serous cancer; gall bladder cancer; bile duct cancer such as for example Klatskin tumour; testicular cancer such as for example seminomas and non-seminomas; lymphoma (lymphosarcoma) such as for example malignant lymphoma, Hodgkin's disease, non-Hodgkin's lymphomas (NHL) such as chronic lymphatic leukaemia, leukaemic reticuloendotheliosis, immunocytoma, plasmocytoma (multiple myeloma), immunoblastoma, Burkitt's lymphoma, T-zone mycosis fungoides, large-cell anaplastic lymphoblastoma and lymphoblastoma; laryngeal cancer such as for example tumours of the vocal cords, supraglottal, glottal and subglottal laryngeal tumours; bone cancer such as for example osteochondroma, chondroma, chondroblastoma, chondromyxoid fibroma, osteoma, osteoid osteoma, osteoblastoma, eosinophilic granuloma, giant cell tumour, chondrosarcoma, osteosarcoma, Ewing's sarcoma, reticulo-sarcoma, plasmocytoma, giant cell tumour, fibrous dysplasia, juvenile bone cysts and aneurysmatic bone cysts; head and neck tumours such as for example tumours of the lips, tongue, floor of the mouth, oral cavity, gums, palate, salivary glands, throat, nasal cavity, paranasal sinuses, larynx and middle ear; liver cancer such as for example liver cell carcinoma or hepatocellular carcinoma (HCC); leukaemias, such as for example acute leukaemias such as acute lymphatic/lymphoblastic leukaemia (ALL), acute myeloid leukaemia (AML); chronic leukaemias such as chronic lymphatic leukaemia (CLL), chronic myeloid leukaemia (CML); stomach cancer or gastric carcinoma such as for example papillary, tubular and mucinous adenocarcinoma, signet ring cell carcinoma, adenosquamous carcinoma, small-cell carcinoma and undifferentiated carcinoma; melanomas such as for example superficially spreading, nodular, lentigo-maligna and acral-lentiginous melanoma; renal cancer such as for example kidney cell carcinoma or hypemephroma or Grawitz's tumour; oesophageal cancer or carcinoma of the oesophagus; penile cancer; prostate cancer; throat cancer or carcinomas of the pharynx such as for example nasopharynx carcinomas, oropharynx carcinomas and hypopharynx carcinomas; retinoblastoma; vaginal cancer or vaginal carcinoma; plate epithelial carcinomas, adenocarcinomas, in situ carcinomas, malignant melanomas and sarcomas; thyroid carcinomas such as for example papillary, follicular and medullary thyroid carcinoma, as well as anaplastic carcinomas; spinalioma, epidormoid carcinoma and plate epithelial carcinoma of the skin; thymomas, cancer of the urethra and cancer of the vulva.

The new compounds may be used for the prevention, short-term or long-term treatment of the above-mentioned diseases, optionally also in combination with radiotherapy or other “state-of-the-art” compounds, such as e.g. cytostatic or cytotoxic substances, cell proliferation inhibitors, anti-angiogenic substances, steroids or antibodies.

The compounds of general formula (1) may be used on their own or in combination with other active substances according to the invention, optionally also in combination with other pharmacologically active substances.

Chemotherapeutic agents which may be administered in combination with the compounds according to the invention include, without being restricted thereto, hormones, hormone analogues and antihormones (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortisone, fluoxymesterone, medroxyprogesterone, octreotide), aromatase inhibitors (e.g. anastrozole, letrozole, liarozole, vorozole, exemestane, atamestane), LHRH agonists and antagonists (e.g. goserelin acetate, luprolide), inhibitors of growth factors (growth factors such as for example platelet derived growth factor and hepatocyte growth factor, inhibitors are for example growth factor antibodies, growth factor receptor antibodies and tyrosinekinase inhibitors, such as for example gefitinib, imatinib, lapatinib, cetuximab (erbitux) and trastuzumab); antimetabolites (e.g. antifolates such as methotrexate, raltitrexed, pyrimidine analogues such as 5-fluorouracil, capecitabin and gemcitabin, purine and adenosine analogues such as mercaptopurine, thioguanine, cladribine and pentostatin, cytarabine, fludarabine); antitumour antibiotics (e.g. anthracyclins such as doxorubicin, daunorubicin, epirubicin and idarubicin, mitomycin-C, bleomycin, dactinomycin, plicamycin, streptozocin); platinum derivatives (e.g. cisplatin, oxaliplatin, carboplatin); alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); antimitotic agents (e.g. Vinca alkaloids such as for example vinblastine, vindesin, vinorelbin and vincristine; and taxanes such as paclitaxel, docetaxel); topoisomerase inhibitors (e.g. epipodophyllotoxins such as for example etoposide and etopophos, teniposide, amsacrin, topotecan, irinotecan, mitoxantron) and various chemotherapeutic agents such as amifostin, anagrelid, clodronat, filgrastin, interferon alpha, leucovorin, rituximab, procarbazine, levamisole, mesna, mitotane, pamidronate and porfimer.

Suitable preparations include for example tablets, capsules, suppositories, solutions—particularly solutions for injection (s.c., i.v., i.m.) and infusion—elixirs, emulsions or dispersible powders. The content of the pharmaceutically active compound(s) should be in the range from 0.1 to 90 wt.-%, preferably 0.5 to 50 wt.-% of the composition as a whole, i.e. in amounts which are sufficient to achieve the dosage range specified below. The doses specified may, if necessary, be given several times a day.

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may also consist of a number of layers to achieve delayed release, possibly using the excipients mentioned above for the tablets.

Syrups containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof. Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

The preparations are administered by the usual methods, preferably by oral or transdermal route, most preferably by oral route. For oral administration the tablets may, of course contain, apart from the abovementioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

For parenteral use, solutions of the active substances with suitable liquid carriers may be used.

The dosage for intravenous use is from 1-1000 mg per hour, preferably between 5 and 500 mg per hour.

However, it may sometimes be necessary to depart from the amounts specified, depending on the body weight, the route of administration, the individual response to the drug, the nature of its formulation and the time or interval over which the drug is administered. Thus, in some cases it may be sufficient to use less than the minimum dose given above, whereas in other cases the upper limit may have to be exceeded. When administering large amounts it may be advisable to divide them up into a number of smaller doses spread over the day.

The formulation examples which follow illustrate the present invention without restricting its scope:

### Examples of Pharmaceutical Formulations

| A) | Tablets | per tablet |
|---|---|---|
| | active substance | 100 mg |
| | lactose | 140 mg |
| | corn starch | 240 mg |
| | polyvinylpyrrolidone | 15 mg |
| | magnesium stearate | 5 mg |
| | | 500 mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

| B) | Tablets | per tablet |
|---|---|---|
| | active substance | 80 mg |
| | lactose | 55 mg |
| | corn starch | 190 mg |
| | microcrystalline cellulose | 35 mg |
| | polyvinylpyrrolidone | 15 mg |
| | sodium-carboxymethyl starch | 23 mg |
| | magnesium stearate | 2 mg |
| | | 400 mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodiumcarboxymethyl starch and the magnesium stearate are added and mixed in and the mixture is compressed to form tablets of a suitable size.

| C) | Ampoule solution | | |
|---|---|---|---|
| | active substance | 50 | mg |
| | sodium chloride | 50 | mg |
| | water for inj. | 5 | ml |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion. The ampoules contain 5 mg, 25 mg and 50 mg of active substance.

1. Compounds of general formula (1) 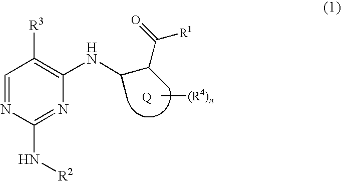
wherein
P Q denotes 5-6 membered heteroaryl, and
R¹ denotes a group selected from among C₁₋₆alkyl, —NR^{c}R^{c} and —OR^{c}, or
R¹ together with a suitable R⁴ forms a 5-7 membered cycloaliphatic ring, which may optionally be substituted by one or more R⁵ and may optionally contain heteroatoms, selected from among N, O and S, and
R² denotes a group, optionally substituted by one or more R⁴, selected from among C₁₋₆alkyl, C₃₋₁₀cycloalkyl, 3-8 membered heterocycloalkyl, C₆₋₁₅aryl and 5-12 membered heteroaryl, and
R³ denotes a group selected from among hydrogen, halogen, —CN, —NO₂, C₁₋₄alkyl, C₁₋₄haloalkyl and —C(O)R^{c}, and
R⁴ and R⁵ each independently denote a group selected from among R^{a}, R^{b} and R^{a} substituted by one or more identical or different R^{c}C and/or R^{b}, and
n denotes 0, 1 or 2, and
each R^{a} is independently selected from among C₁₋₆alkyl, C₃₋₁₀cycloalkyl, C₄₋₁₆cycloalkylalkyl, C₆₋₁₀aryl, C₇₋₁₆arylalkyl, 2-6 membered heteroalkyl, 3-8 membered heterocycloalkyl, 4-14 membered heterocycloalkylalkyl, 5-12 membered heteroaryl and 6-18 membered heteroarylalkyl, and
each R^{b} is a suitable group and each independently selected from among ═O, —OR^{c}, C₁₋₃haloalkyloxy, —OCF₃, ═S, —SR^{c}, ═NR^{c}, ═NOR^{c}, —NR^{c}R^{c}, halogen, —CF₃, —CN, —NC, —OCN, —SCN, —NO₂, —S(O)R^{c}, —S(O)₂R^{c}, —S(O)₂OR^{c}, —S(O)NR^{c}R^{c}, —S(O)₂NR^{c}R^{c}, —OS(O)R^{c}, —OS(O)₂R^{c}, —OS(O)₂OR^{c}, —OS(O)₂NR^{c}R^{c}, —C(O)R^{c}, —C(O)OR^{c}, —C(O)NR^{c}R^{c}, —CN(R^{f})NR^{c}R^{c}, —CN(OH)R^{c}, —CN(OH)NR^{c}R^{c}, —OC(O)R^{c}, —OC(O)OR^{c}, —OC(O)NR^{c}R^{c}, —OCN(R^{f})NR^{c}R^{c}, —N(R^{f})C(O)R^{c}, —N(R^{f})C(S)R^{c}, —N(R^{f})S(O)₂R^{c}, —N(R^{f})C(O)OR^{c}, —N(R^{f})C(O)NR^{c}R^{c}, —[N(R^{f})C(O)]₂R^{c}, —N[C(O)]₂R^{c}, —N[C(O)]₂OR^{c}, —[N(R^{f})C(O)]₂OR^{c} and —N(R^{f})CN(R^{f})NR^{c}R^{c}, and
each R^{c} independently denotes hydrogen or a group optionally substituted by one or more identical or different R^{d} and/or R^{e} selected from among C₁₋₆alkyl, C₃₋₁₀cycloalkyl, C₄₋₁₁cycloalkylalkyl, C₆₋₁₀aryl, C₇₋₁₆arylalkyl, 2-6 membered heteroalkyl, 3-8 membered heterocycloalkyl, 4-14 membered heterocycloalkylalkyl, 5-12 membered heteroaryl and 6-18 membered heteroarylalkyl, and
each R^{d} independently denotes hydrogen or a group optionally substituted by one or more identical or different R^{e} and/or R^{f} selected from among C₁₋₆alkyl, C₃₋₈cycloalkyl, C₄₋₁₁cycloalkylalkyl, C₆₋₁₀aryl, C₇₋₁₆arylalkyl, 2-6 membered heteroalkyl, 3-8 membered heterocycloalkyl, 4-14 membered heterocycloalkylalkyl, 5-12 membered heteroaryl and 6-18 membered heteroarylalkyl, and
each R^{e} is a suitable group and each independently selected from among ═O, —OR^{f}, C₁₋₃haloalkyloxy, —OCF₃, ═S, —SR^{f}, ═NR^{f}, ═NOR^{f}, —NR^{f}R^{f}, halogen, —CF₃, —CN, —NC, —OCN, —SCN, —NO₂, —S(O)R^{f}, —S(O)₂R^{f}, —S(O)₂OR^{f}, —S(O)NR^{f}R^{f}, —S(O)₂NR^{f}R^{f}, —OS(O)R^{f}, —OS(O)₂R^{f}, —OS(O)₂OR^{f}, —OS(O)₂NR^{f}R^{f}, —C(O)R^{f}, —C(O)OR^{f}, —C(O)NR^{f}R^{f}, —CN(R^{g})NR^{f}R^{f}, —CN(OH)R^{f}, —C(NOH)NR^{f}R^{f}, —OC(O)R^{f}, —OC(O)OR^{f}, —OC(O)NR^{f}R^{f}, —OCN(R^{g})NR^{f}R^{f}, —N(R^{g})C(O)R^{f}, —N(R^{g})C(S)R^{f}, —N(R^{g})S(O)₂R^{f}, —N(R^{d})C(O)OR^{f}, —N(R^{g})C(O)NR^{f}R^{f}, and —N(R^{g})CN(R^{f})NR^{f}R^{f}, and
each R^{f} independently denotes hydrogen or a group optionally substituted by one or more identical or different R^{g} selected from among C₁₋₆alkyl, C₃₋₈cycloalkyl, C₄₋₁₁cycloalkylalkyl, C₆₋₁₀aryl, C₇₋₁₆arylalkyl, 2-6 membered heteroalkyl, 3-8 membered heterocycloalkyl, 4-14 membered heterocycloalkylalkyl, 5-12 membered heteroaryl and 6-18 membered heteroarylalkyl, and
each R^{g} independently denotes hydrogen, C₁₋₆alkyl, C₃₋₈cycloalkyl, C₄₋₁₁cycloalkylalkyl, C₆₋₁₀aryl, C₇₋₁₆arylalkyl, 2-6 membered heteroalkyl, 3-8 membered heterocycloalkyl, 4-14 membered heterocycloalkyl, 5-12 membered heteroaryl and 6-18 membered heteroarylalkyl,
optionally in the form of the tautomers, the racemates, the enantiomers, the diastereomers and the mixtures thereof, and optionally the pharmacologically acceptable salts thereof.

2. Compounds according to claim 1, wherein R³ denotes halogen or —CF₃.

3. Compounds according to claim 1, wherein R³ denotes —CF₃.

4. Compounds according to claim 1, wherein Q is selected from among thiophene, pyrrole, pyrazole and imidazole, optionally substituted by one or more R⁴.

5. Compounds according to claim 1, wherein Q is selected from among 1,2,3,4-tetrahydro-pyrrolo[1,2-a]pyrazine and 4,5,6,7-tetrahydro-pyrazolo[1,5-a]pyrazine, optionally substituted by one or more R⁵.

6. Compounds according to claim 1, wherein R² denotes a group, optionally substituted by one or more R⁴, selected from among C₆₋₁₅aryl and 5-12 membered heteroaryl.

7. Compounds, or the pharmaceutically effective salts thereof, according to claim 1 for use as pharmaceutical compositions.

8. Compounds, or the pharmaceutically effective salts thereof, according to claim 1 for preparing a pharmaceutical composition with an antiproliferative activity.

9. Pharmaceutical preparations, containing as active substance one or more compounds of general formula (1) according to claim 1 or the pharmacologically acceptable salts thereof, optionally in combination with conventional excipients and/or carriers.

10. Use of compounds of general formula (1) according to claim 1 for preparing a pharmaceutical composition for the treatment and/or prevention of cancer, infections, inflammations and autoimmune diseases.

11. Pharmaceutical preparation comprising a compound of general formula (1) according to claim 1, optionally in the form of the tautomers, the racemates, the enantiomers, the diastereomers and the mixtures thereof, and optionally the pharmacologically acceptable salts thereof and at least one further cytostatic or cytotoxic active substance different from formula (1).
